(19) United States
(12) Patent Application Publication
(10) Pub. No.: US 2024/0285890 A1
(43) Pub. Date: Aug. 29, 2024

(54) NASOPHARYNGEAL AIRWAY DEVICE

(71) Applicant: InnoVital, LLC, Calverton, MD (US)

(72) Inventors: Amit N. Shah, North Potomac, MD (US); Curt S. Kothera, Laurel, MD (US); Thomas E. Pillsbury, Frederick, MD (US); Pablo J. Sztein, Silver Spring, MD (US)

(21) Appl. No.: 18/587,298

(22) Filed: Feb. 26, 2024

Related U.S. Application Data
(60) Provisional application No. 63/487,296, filed on Feb. 28, 2023.

(51) Int. Cl. A61M 16/04

(52) U.S. Cl. CPC A61M 16/0461; A61M 2205/0216; A61M 2209/088; A61M 2210/0618; A61M 2210/065

(57) ABSTRACT

A nasopharyngeal airway device comprising a flexible tubular body with an offset opening at its distal end configured for placement in a patient's pharynx via insertion through a nostril and extending to another opening outside the nose of the patient to serve as a temporary airway. The device includes various features that make it easy-to-use and facilitate its use, thereby enabling improved patient outcomes.

### CROSS-REFERENCE TO RELATED APPLICATION(S)

The present application derives priority from U.S. Provisional Patent Application No. 63/487,296 filed 28 Feb. 2023.

### BACKGROUND OF THE INVENTION

### 1. Field of the Invention

The present invention relates to a device that is placed in the nose of a patient to serve as a temporary airway.

### DESCRIPTION OF THE BACKGROUND

The nasopharyngeal airway (NPA) is a critical tool for airway management across a range of clinical settings. The NPA is designed to provide a patent ventilatory passage in a patient who is unconscious, of otherwise altered mental status, or sedated, including in scenarios where the ventilatory passage would otherwise be or become obstructed. NPA insertion, through the nostril, requires minimal skill and experience and may be performed in patients who would not tolerate other airway procedures such as oropharyngeal airway or extraglottic device placement without sedating or paralytic medication. Of note, endoscopic evaluations of sleep apnea patients demonstrate that a properly placed NPA can prevent airway obstruction at multiple levels. Victores A J, Olson K, Takashima M., Interventional Drug-Induced Sleep Endoscopy: A Novel Technique to Guide Surgical Planning for Obstructive Sleep Apnea, J. Clin Sleep Med., 13(2):169-174 (2017). These benefits coupled with relatively rare procedural complications make NPA placement a staple of airway management. There are, however, multiple common ways in which the NPA can fail or perform sub-optimally, thereby offering opportunities for innovation and improvement.

The current procedural approach, which utilizes NPA devices that have a fixed length for each diameter and base device sizing decisions on unreliable nostril width and nares-earlobe length assessments, often results in improper NPA tip placement and suboptimal NPA function. Roberts K, Whalley H, Bleetman A, The Nasopharyngeal Airway: Dispelling Myths And Establishing The Facts, Emerg Med J., 22:394-396 (2005); Sareen B, Kapur A, Gupta S K et. al., Clinical Evaluation Of Nares—Vocal Cord Distance And Its Correlation With Various External Body Parameters, Indian J Anaesth, 59(4):212-215 (2015); Stoneham M D, The Nasopharyngeal Airway. Assessment of Position by Fibreoptic Laryngoscopy Anaesthesia, July; 48(7): 575-80 (1993).

Once a device is chosen, it is simply inserted to its full length until a feature near its proximal end abuts the nose of a patient. During this full insertion step (through the right nostril), the user also typically must rotate the device such that the distal opening is oriented toward the septum.

Multiple authors have described a relationship between nares-epiglottis length with height and have suggested NPA depth markings and sizing charts to enhance accuracy of NPA insertion. Roberts supra; Sareen supra; Stoneham supra; Tseng W-C, Lin W-L, Cherng C-H, Estimation Of Nares-To-Epiglottis Distance For Selecting An Appropriate Nasopharyngeal Airway, Medicine 98:10 (2019).

An improved device should facilitate NPA tip placement at an appropriate depth, preventing soft tissue collapse and airway obstruction and enabling ventilation. As an example, in anesthetized patients, an NPA seated with its distal end falling one centimeter superior to the tip of the epiglottis has been shown to address multiple points of potential airway obstruction, including by separating the soft palate from the posterior oropharyngeal wall. An NPA seated at this level is also not so deep as to enter the larynx or lodge in the vallecula (which results in paradoxical airway obstruction). Roberts supra; Stoneham supra.

Instead of having multiple diameters and lengths of NPA devices that the provider must choose between, an adaptable one-size-fits-all (or one-size-fits-most) NPA could greatly simplify its use. Color-coded bands corresponding to ranges in height have proven effective in other areas of patient treatment. Hoyle J D Jr, Ekblad G. et. al., Methods Used to Obtain Pediatric Patient Weights, Their Accuracy and Associated Drug Dosing Errors in 142 Simulated Prehospital Pediatric Patient Encounters., Prehosp Emerg Care., 2021:1-8. Such bands might be applied to an improved NPA as a means of gauging insertion depth on the universal device.

For example, three color zones may be employed with printed height ranges on the device itself (e.g., patients gauged to be 5′10″ and above would be assigned to one of three color zones) to enhance NPA tip positioning. Other approaches to gauging accuracy of placement—e.g., assessing flow through the NPA at various positions—may also be used to optimize NPA placement.

Some currently marketed NPA devices come with an adjustable ring that can be used to effectively reduce the inserted length of the NPA tube by sliding the ring against the patient's nose before the NPA is fully inserted. These rings can be quite ineffective, however, because nothing holds them in place. Their incapacity for maintaining a shorter inserted length is further exacerbated by the fact that the NPA is lubricated during insertion, which reduces the friction between the tube and adjustable ring.

Even if the NPA is optimally placed, airway obstruction can still occur with traditional NPAs due in part to the inability of existing NPAs to resist collapse at multiple levels. Stoneham supra; Victores A J supra. Collapse of the NPA ventilatory conduit and ensuring airway obstruction in an obtunded, unattended casualty can lead to severe consequences-specifically, obstructed ventilation, hypercarbia, acidosis, hypoxia, and ultimately cardiac arrest. An improved NPA would at a minimum reduce the prevalence of collapse. Part of the challenge in such an enhancement to an NPA is the competing mechanical requirements of a device that must be soft and flexible enough to be easily and atraumatically placed, yet when seated is resistant to collapse/reduction in internal diameter.

There is also a documented under-utilization of NPAs in prehospital trauma care. Alarmingly, cricothyrotomy (the much more invasive procedure of surgical airway creation which carries a prehospital placement failure rate of 33%) is performed at nearly the same frequency as NPA insertion, including in clinical scenarios where NPA placement may have been the more appropriate intervention. Mabry R L, An Analysis Of Battlefield Cricothyrotomy In Iraq & Afghanistan, J Spec Oper Med., 12(1):17-23 (2021).

Exact causes for the NPA under-utilization are not clear, but likely include a lack of understanding of how NPAs should be utilized in the field. Consequently, there remains a need for an NPA that requires less field expertise.

The present invention advances the existing state of the art for nasopharyngeal airways and adds features that aid the operator in obtaining optimal positioning, maintaining patency, and enabling continuous ventilatory monitoring.

### SUMMARY OF THE INVENTION

According to an embodiment of the invention, a nasopharyngeal airway (NPA) is herein disclosed that is an easy-to-use device designed to facilitate correct use. The NPA is generally a flexible tubular structure of circular cross-section, having at least one distal opening and a proximal opening, and may have a slight curvature along its length axis. It may have a plurality of other features to achieve its design goal, including those that are listed below:
a. Inline distal opening. The primary distal opening may be inline, preferably along the inside of the curvature of the NPA, such that when the provider inserts the NPA in either nostril following the curvature of the device, the bevel of the distal opening is oriented in maximal alignment with the glottis of the patient. This would make it such that the provider does not have to specifically rotate the bevel (e.g., toward the septum) as a first step.
b. Large primary distal opening. The distal opening may be larger than that of most existing NPAs to promote better air flow, with the cut of the bevel at a shallower angle and located at a different position of the tube tip.
c. Multiple additional distal fenestrations. There may be at least one additional, preferably large fenestration placed near the distal end of the device, but not generally larger than the primary distal opening. Fenestrations may be varied in their clocking/placement around the circumference of the tube for redundancy and to help ensure that a ventilatory path exists even in instances of suboptimal placement (e.g., if the distal tip is placed in the vallecula). Distal fenestrations may also be (e.g., equidistantly) spaced longitudinally (i.e., along the long axis of the device) and may be shaped to promote flow, maintain structural integrity of the tube, and reduce the likelihood of blockage.
d. Atraumatic tip. The distal tip of the NPA may be designed to be soft for atraumatic insertion. Specifically, the wall thickness near the bevel region of the primary distal opening may be less than that of the body of the device, reducing its stiffness. The tip is also preferably rounded. These features allow for the tip to yield and flex, thereby enabling atraumatic delivery through the nasal passage of a patient.
e. Adjustable insertion depth zones. Traditional approaches to NPA sizing and positioning rely on unreliable facial measurements. In contrast, the anatomic measurement most relevant to NPA sizing-nares epiglottis length-correlates best with patient height. The subject invention may contain a specialized flange that allows depth adjustment including across labeled zones and easy securement at the selected depth. Adjustment zones based on height may be indicated on the tube itself via physical features or markings. Alternatively, markings to guide stepwise NPA adjustment to maximize flow, as sensed through simply listening to air flow through the NPA or via a flow sensor, may be included on the device. In either of the above embodiments of an adjustable NPA, the tube length of the NPA is longer than typical NPA devices such that it may be used as a one-size-fits-most NPA leveraging the adjustable flange, with some extra length expected to extend from the nostril of a shorter patient.
f. Adjustable flange. The subject invention may have a flange positioned initially toward the proximal end of the device that is manually adjustable to set the selected (i.e., optimal) depth of NPA insertion and preferably maintain this set length during use in the patient. The adjustable flange may serve these functions even in the presence of lubricants. The NPA body and/or flange may have features that facilitate the noted depth setting of the flange along the NPA body including but not limited to twist-lock features, friction lock features, embossments, squeeze-temporary shape change features, and tapered interface areas.
g. Securement features. The adjustable flange may accommodate various methods to secure the inserted NPA device to the patient, depending on availability and the patient scenario. Examples include but are not limited to a ribbon around the head, elastic ear straps, and taping. One exemplary approach to accommodating such methods of securement, though not intended to be limiting, is the presence of at least one slot in the flange through which securing devices can be threaded. The flange may also contain features that help affix the device to the patient's nose or face, such as a clip, spring, clamp, or string.
h. Stiffness. The subject invention may be manufactured from a material that has an appropriate balance of stiffness and wall thickness to ensure that the NPA body resists collapse from pressure due to anatomic structures (e.g., tongue) but also enables smooth, atraumatic placement and comfort after placement.
i. Compatibility with bag-valve. Ventilation of a patient is often accomplished via a bag-valve-mask (BVM). Bag-valves have a standard connector that attaches to the adapter type commonly found on masks and endotracheal tubes. The subject invention may feature a proximal tube end that is the same size and shape of this adapter to ensure compatibility with bag-valves. This “proximal adapter end” may be formed from the NPA tube material itself or be a secondary component possibly of a different material that is assembled into or removably attached to the subject device. The proximal adapter end may also be compatible with a variety of accessories and monitoring devices such as the EMMA capnograph.
j. Placement guide and continuous respiratory check. Misting/condensation of airway tubes has long been used as an indicator of flow/ventilation. At least the proximal end of the subject device may be designed for at least semi-transparency or translucency such that breath misting can be more readily visualized by the provider. Misting can be used to assess appropriate placement, as well as for ongoing patient ventilatory monitoring. Aside from at least semi-transparency of at least a proximal section of the NPA tube, other features that may be included to facilitate this visualization include but are not limited to optimizing tube material or adding a coating (such as a hydrophilic coating) to the inside surface of the tube to enhance/accentuate condensation, optimizing the surface finish of the tube surface to enhance/accentuate condensation, choosing a semi-transparent color that highlights the condensation, including a magnification effect along a proximal section of the tube, and introducing a material that is dynamically responsive to carbon dioxide, pH, moisture, and/or temperature. Mechanical flow sensors also enable such a check of spontaneous ventilation and may include a part that visibly moves with each inspiratory/expiratory cycle of a patient.

The present invention is described in greater detail in the detailed description of the invention, and the appended drawings. Additional features and advantages of the invention will be set forth in the description that follows, will be apparent from the description, or may be learned by using the invention.

### BRIEF DESCRIPTION OF THE DRAWINGS

Other objects, features, and advantages of the present invention will become more apparent from the following detailed description of the preferred embodiments and certain modifications thereof when taken together with the accompanying drawings in which:

FIG. 1 depicts a perspective view of a preferred embodiment of the nasopharyngeal airway device of the present invention.

FIG. 2 shows a perspective view of a preferred embodiment of the tube body part of the nasopharyngeal airway device of the present invention.

FIG. 3 shows a close-up view of the proximal end of the tube body of preferred embodiment of the nasopharyngeal airway device of the present invention.

FIG. 4 shows a perspective view of an embodiment of the tube body with a partially radial fixed flange.

FIG. 5 shows a close-up view of the distal end of preferred embodiment of the nasopharyngeal airway device of the present invention.

FIG. 6 shows a perspective view of a preferred embodiment of the adjustable flange part of the nasopharyngeal airway device of the present invention.

FIG. 7 shows a rear perspective view of a preferred embodiment of the nasopharyngeal airway device of the present invention.

FIG. 8 shows a perspective view of an embodiment of the adjustable flange part with additional gripping features for the user.

FIG. 9 shows a perspective view of an alternate embodiment of the adjustable flange part with additional gripping features for the user.

FIG. 10 shows a perspective view of an embodiment of the adjustable flange part with an additional securement feature for the patient.

### DETAILED DESCRIPTION OF THE PREFERRED EMBODIMENT

Reference will now be made in detail to preferred embodiments of the present invention, examples of which are illustrated in the accompanying drawings. Wherever possible, the same reference numbers will be used throughout the drawings to refer to the same or like parts.

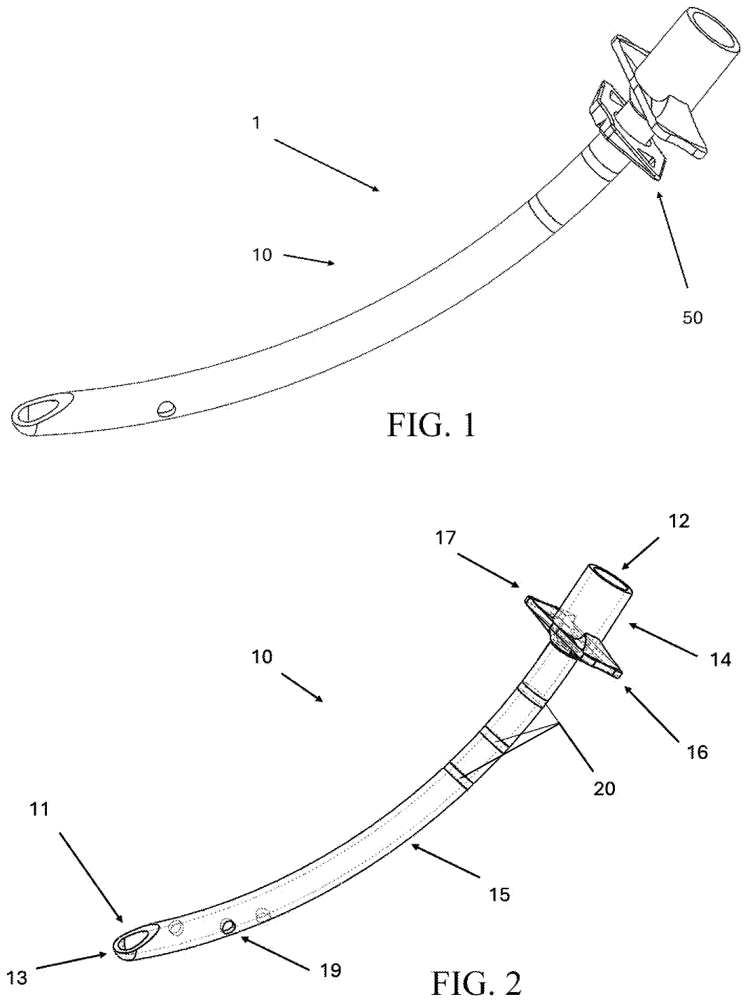

The present invention is a nasopharyngeal airway (NPA) device. As shown in FIG. 1, a preferred embodiment of NPA device 1 is comprised of two parts: (1) tube body 10; and (2) adjustable flange 50. Both of these parts 10, 50 are preferably made of elastomeric materials, and more preferably made of silicone material. However, the two parts are preferably made of materials of different stiffnesses, with tube body 10 generally made of a softer (lower durometer) material than adjustable flange 50. Adjustable flange 50 is formed with a collar sized to fit slidably over tube body 10 to yield a fully-assembled NPA device 1. In addition to these two parts 10, 50, the preferred embodiment also includes at least one securing strap 70 that is affixed to adjustable flange 50. More details of the individual parts of device 1 will be described below.

In reference to FIG. 2, NPA tube body 10 is generally a flexible cylindrical structure of annular cross-section, with a distal opening 11 at one end, a proximal opening 12 at the other end, and a stepped transition from the larger proximal opening 12 to smaller-diameter distal opening 11. Tube body 10 preferably has a resting shape-memory curvature along its length axis. The material stiffness and tube wall thickness are such that the tube body 10 does not collapse or kink under loads of anatomic structures, yet also soft enough that the tube body 10 can be inserted through a patient's nose atraumatically and rest in the patient comfortably.

Distal opening 11 is preferably offset from a longitudinal center of the flexible tubular body 10, formed at an angle to the circular cross-section of the tubular body 10 so as to form an oblong aperture facing inward toward the inside curvature. Preferably, the annulus of the tubular body 10 at the first distal end circumscribes a circular interior, and the oblong distal opening 11 has an area greater than the local circular interior at the distal end of tubular body 10. Further, while the distal opening 11 occupies the interior tip of tube body 10, the exterior tip remains and is preferably rounded to aid with atraumatic insertion. The tube wall thickness at and near this distal rounded tip 13 may also be less than that of the majority of the tube body to increase its softness for atraumatic placement.

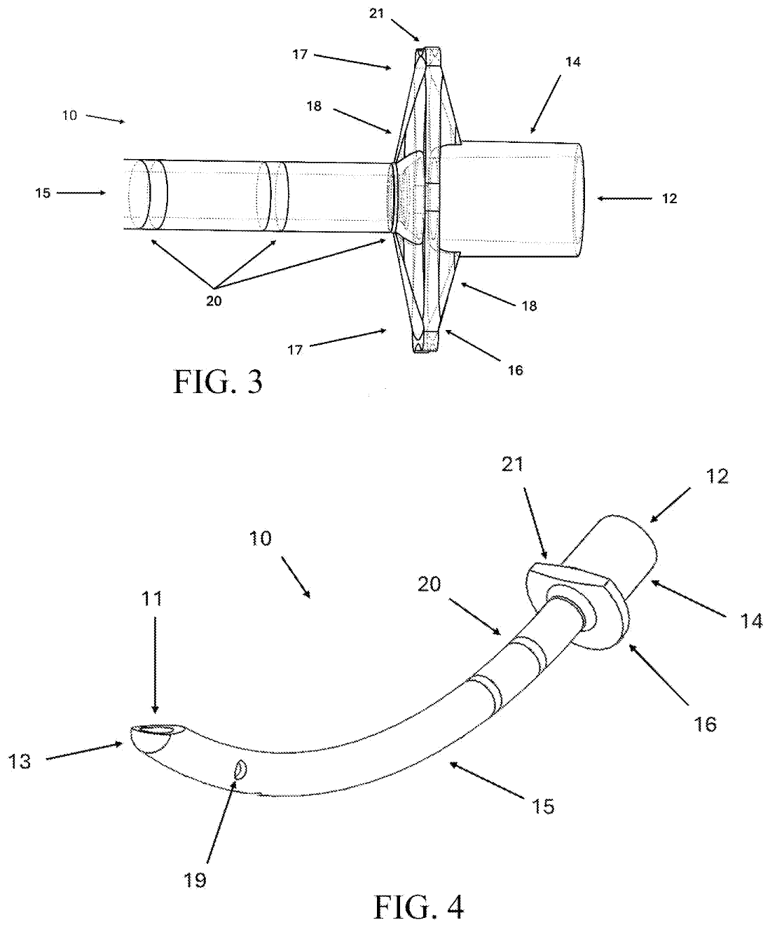

FIG. 3 shows more detail of the proximal end of tube body 10 configured as a relatively short tubular cylinder with an annular cross-section at the proximal end and having a proximal opening 12 of larger diameter 14 than the circular interior of the second proximal end of main tube body 15, and preferably sized and shaped like a conventional endotracheal tube connector (e.g., a 15 mm connector). There is preferably a stepped transition from the proximal end of tube body 10 (from larger proximal end diameter 14) to main tube body diameter 15, though this is not meant to be limiting as any form of transition could be present here within the spirit of the invention. At the point of diameter change, a fixed flange 16 is located and preferably formed of the same material as NPA tube body 10. Any form of flange could be located here within the spirit of the invention, though the preferred shape has lateral extensions 17. Fixed flange 16 may also have grip features 21, such as depressions, to promote gripping by a user, though additional extensions for this purpose could also be included within the invention. Then, generally, distal to fixed flange 16 is main tube body 15 of the NPA and proximal to fixed flange 16 is connector end 14. Main tube body 15 is preferably 165 mm in length from fixed flange 16 to distal tip 13, though it could generally range from 120 mm to 190 mm for adult patients.

Having proximal opening 12 preferably shaped like an endotracheal tube connector offers advantages to device 1. These include, but are not limited to, enabling attachment of a bag-valve for direct ventilation via device 1, enabling attachment of diagnostic equipment such as capnographs, and enabling attachment of flow sensors that provide continuous feedback of a patient's spontaneous ventilation. Such types of sensors may include mechanical devices with parts that move in relation to a patient's inspiration and expiration, and chemical or colorimetry devices that change color in response to a patient's breathing.

Aside from preferred lateral extensions 17 of fixed flange 16, other embodiments could also include other functional features. Examples include, but are not limited to, a circular/radial flange segment all or partially around the circumference, stiffening ribs 18, and an extension generally along the length axis and partially outward, oriented toward the proximal tip of the connector end. FIG. 4 shows one such embodiment where fixed flange 16 is radial around most of tube body 10 but truncated along a flat section 21 along the inside of the curvature. Flat section 21 provides for more comfortable resting of device 1 against a patient's upper lip and also provides some asymmetry to the design of tube body 10 that serves as an indication to the user of which direction primary distal opening 11 is oriented inside the patient.

While tube body 10 is preferably formed of a single material in a single process, another embodiment may form tube body 10 as an assembly of more than one part of the same material with the assembly occurring preferably at the location of fixed flange 16, or as an assembly of more than one part of different materials, again with the assembly occurring preferably at the location of fixed flange 16. In any such embodiment, the point of attachment between the different parts is preferably seamless such that the result appears as a single unit. Chemical or mechanical means of attachment are preferred.

Regarding materials, at least the proximal end of tube body 10 is preferably made of a material that is either transparent or translucent, such that condensation from a patient's breathing would be visible to an observer through device 1. In embodiments where tube body 10 is formed of a single material, then that material preferably has a so-called “misting” visibility wherein breath moisture condensation is visible along the transparent/translucent walls. In embodiments where tube body 10 is formed of more than one material, then at least the proximal end of tube body 10 is preferably formed of a transparent or translucent material, since an observer would only be able to see the misting in the portion of device 1 that extends outward from a patient's nose. This would enable continuous feedback for visible monitoring of a patient's breathing or spontaneous ventilation when device 1 was inserted, when the atmospheric conditions permit condensation to form.

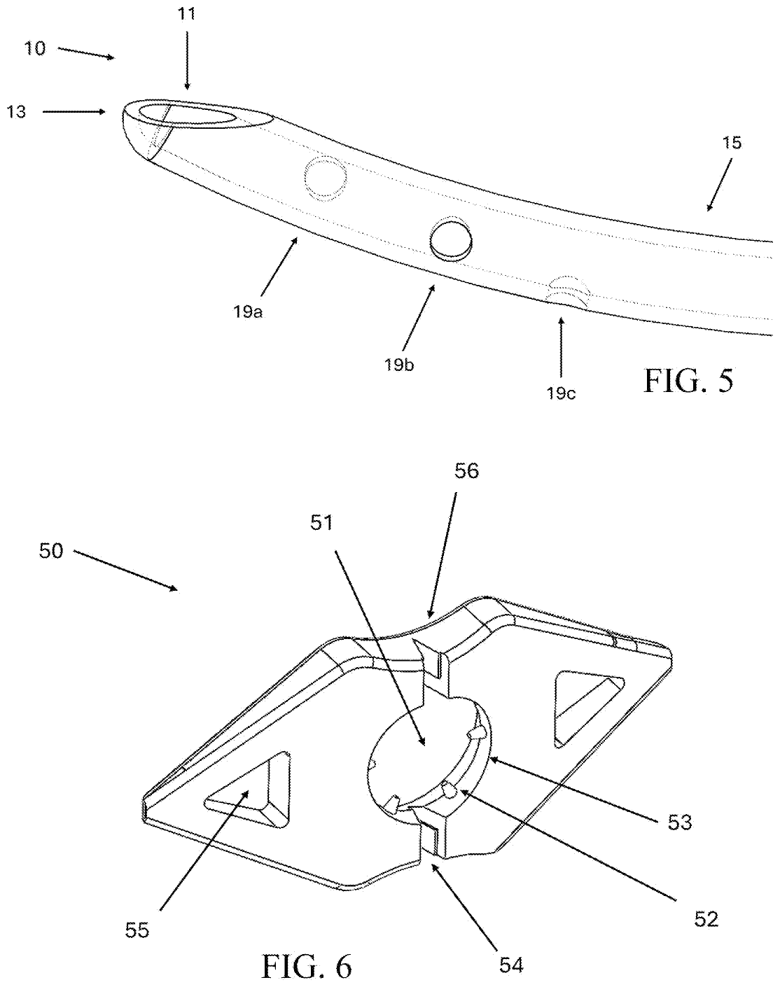

FIG. 5 shows a close-up view of the distal end of tube body 10. Near the distal end of the tube body, but proximal to primary distal opening 11, the preferred embodiment has three secondary openings 19a, 19b, 19c, each of which is smaller than primary distal opening 11. These secondary openings 19 are preferably of the same diameter as each other, no larger than the inner diameter of main tube body 15, and provide access through the side walls of main tube body 15. Secondary openings 19 are preferably spaced circumferentially (i.e., radially oriented) and are angularly offset (e.g., 90 degrees from each other). In addition, secondary openings 19 are longitudinally spaced from each other and the primary distal opening (e.g., more than one diameter away from each other). Note that the number of secondary openings 19, their size, spacing, and orientation is not meant to be limiting in the invention.

Referring back to FIGS. 2-3, near the proximal end of main tube body 15, but distal to fixed flange 16, the preferred embodiment has a plurality of indicators 20, preferably three, longitudinally spaced from each other and visible from various angles around the device. How the indicators 20 are formed is not limiting to the invention and could include options such as external markings (e.g., pad printing, laser etching, silicone painting), indentations in the tube material, embossments in the tube material, or differences in material finish; nor is the makeup of the indicators (e.g., solid lines/rings, dashed lines, dotted lines) meant to limit the invention. The purpose of indicator markings 20 in the invention is to provide the user with a set of distinct locations for re-positioning adjustable flange 50, which can be used with the stepwise approach of the instructions for use of the device to incrementally decrease the length of tube inserted in the patient's nose in searching for an optimal insertion depth. Adjustable flange 50 generally starts at the proximal-most position indicated by markings 20 (e.g., nearest to fixed flange 16). Indicator markings 20 are preferably spaced 15 mm apart along the length of main tube body 15, though they could range from 5 mm to 25 mm for an adult-size NPA.

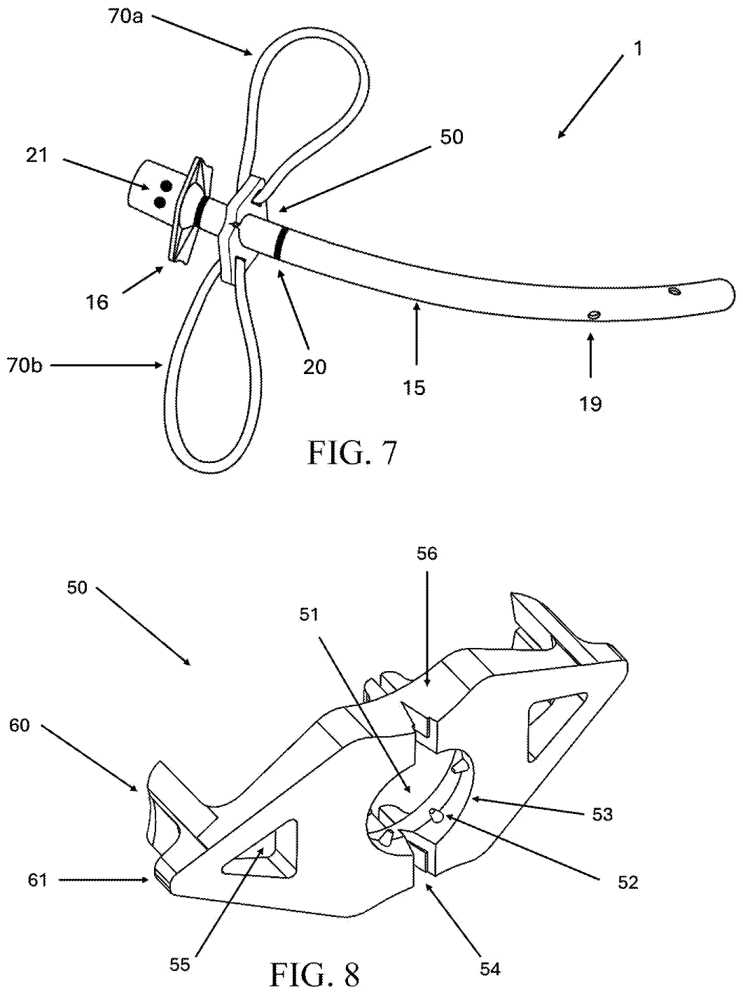

As seen in FIG. 6, adjustable flange 50 is generally a thin rectangular or diamond shaped part with a hole 51 through it to enable it to slide along main tube body 15 of device 1. Flange hole 51 fits snuggly with main tube body 15 and this snug fit may be augmented with protrusions 52 that extend radially inward from inside face 53 of hole 51. The size, shape, and number of protrusions 52 is not meant to be limiting to the invention, but the preferred embodiment has four frusto-conical protrusions 52. Protrusions 52 provide additional grip to hold adjustable flange 50 position relative to tube main body 15. Providing yet another means of grip is the pinching force exerted on main tube body 15 by adjustable flange 50, which is enhanced by cutout notches 54 in the center of flange 50. These cutouts 54 allow adjustable flange 50 to bend (i.e., toward the nose) when force is applied to them. As seen in FIG. 7, device 1 is secured to the patient via at least one securement strap 70 attached on opposing sides of flange 50 that draw the device toward the patient and apply force causing adjustable flange 50 to bend at cutouts 54. Adjustable flange 50 preferably has cutouts 55 in the lateral extension areas to facilitate two securement strap(s) 70a, 70b through means of tying, fastening, or using elastic ear loops. Because of the increase in holding force between adjustable flange 50 and main tube body 15 when flange 50 is bent or flexed toward cutouts 54, adjustable flange 50 may also include depressions 56 as locations for the user to preferentially hold flange 50 without causing it to bend. Depressions 56 are preferably centrally located, in line with the bend axis created by cutouts 54, and sized and shaped to provide some positive resistance for a user's finger tips.

The specific type of strap(s) used for securement strap(s) 70a, 70b is not meant to be limiting to the invention, though the invention calls for a means of securement to the patient via a strap-like device that is attached to adjustable flange 50. Such a strap is preferably elastomeric or of a similar design that allows sufficient length change to accommodate a variety of potential patient head circumferences, including potentially with a helmet on. Elastic ear loops, like what is commonly found on surgical masks are one such option for securement, with one loop being affixed to each lateral side of the adjustable flange 50. An alternate embodiment may use a single elastic-like strap that is initially affixed to one side of the adjustable flange at one end and free at the other end, such that the free end can be passed behind/around the patient's head and then attached to the opposite side of the adjustable flange. Such an attachment may be accomplished by any number of means, though preferably would be a hook-like device.

FIG. 7 shows a rear perspective view of an embodiment of the invention where securement straps 70 are ear loops that are attached to each cutout 55 of adjustable flange 50. Note also that this embodiment has indica 21 that provides information to the user of the orientation of device 1 when it is inserted into a patient since the distal opening 11 and secondary openings 19 will no longer be visible.

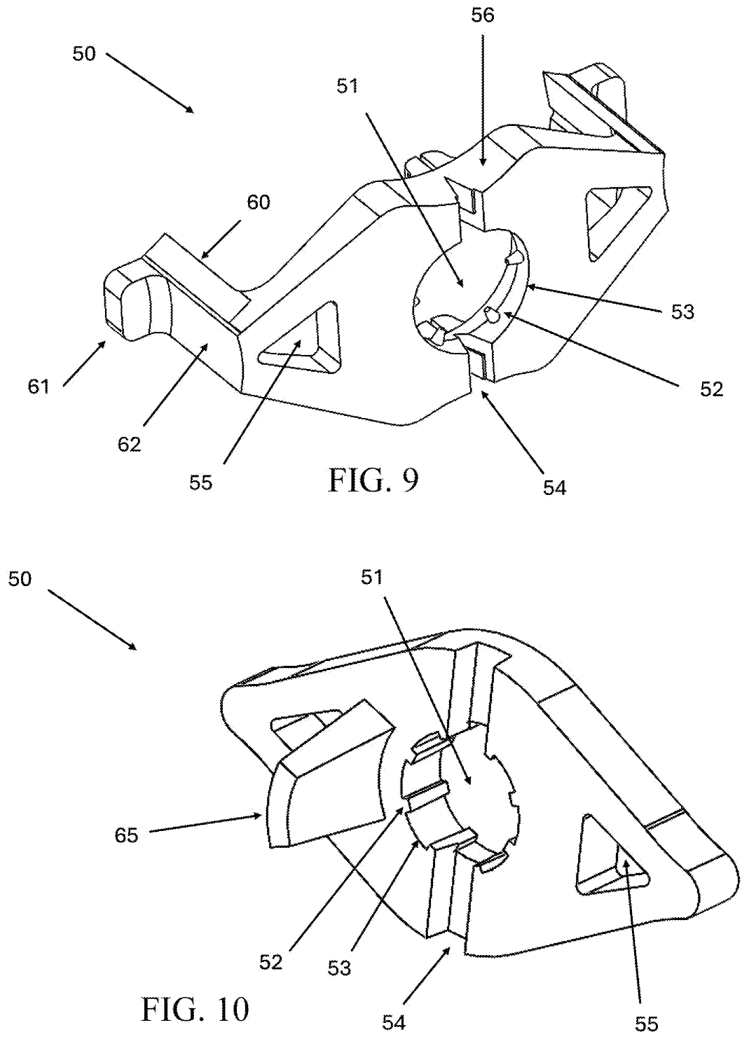

FIG. 8 shows an embodiment of adjustable flange 50 with two additional pairs of features intended to provide contact area to the user in a location that reduces the bending potential of the part. Gripping extensions 60 preferably extend proximally from the body of flange 50 and in an axial orientation (relative to main tube body 15). A flange 50 with such gripping extensions 60 may also have lateral extensions 61 generally collocated with gripping extensions 60 to provide additional means of gripping flange 50, particularly in the presence of lubricants. Note that the placement of lateral extensions 61 relative to gripping extensions 60 can vary within the spirit of the invention, such as that shown in FIG. 9. Additionally, gripping extensions 60 may have depressions 62 to promote secure finger placement, similar to depressions 56.

FIG. 10 shows another embodiment of adjustable flange 50 that includes a different generally axially oriented extension, securing extension 65. Securing extension 65 is preferably more centrally located near hole 51 and extends distally. There is also preferably only one securing extension 65 on adjustable flange 50. The primary purpose of securing extension 65 is to provide additional means of securement to the patient, such as to the nose. In this way, securing extension 65 acts somewhat like a nose clip and there is preferably only one so it can be applicable to a device 1 inserted in either patient nostril by simply rotating it about the length axis of main tube body 15.

The preferred method of use for device 1 is as described below. Note that the starting position of adjustable flange 50 is in the proximal-most position along main tube body 15, just distal to fixed flange 16.

First, the user is to lubricate main tube body 15 with sterile, water-based gel. Then the user is to orient device 1 so distal tip 13 is pointing at the patient's nose and primary distal opening 11 is toward the mouth.

Second, the user is to gently lift the tip of the patient's nose to open the nostril, and then insert device 1 on a path generally toward the patient's ear lobe. Since this is a one-size-fits-most device, the initial insertion should be a full insertion such that adjustable flange 50 is against the nose. Also, since primary distal opening 11 is along the insert edge of tube curvature, it does not generally matter which nostril device 1 is inserted in. If the patient is observed to gag as a result of full insertion, the user should back device 1 out slightly so the gagging stops, and then slide adjustable flange 50 against the nose while keeping tube 10 in its backed out position.

Third, the user is to assess air flow through device 1 for five to six breaths. This could be accomplished by any number of means, such as listening, feeling, capnography, or other mechanical flow indicators.

If flow through device 1 is not observed or very weak, the user is to incrementally adjust (decrease) the inserted depth. This is done by holding adjustable flange 50 against the patient's nose, preferably in a manner that does not cause flange 50 to bend, as bending flange 50 causes it to pinch main tube body 15, which increases the force required to slide main tube body 15 relative to adjustable flange 50. While holding flange 50, the user is to withdraw the fully inserted main tube body 15 by one marking 20. That is, the user is to pull out tube body 10 until the next marking 20 aligns with adjustable flange 50.

At this point, the user is to re-assess air flow through device 1 for five to six breaths. The same method of assessment used previously is acceptable again. If flow is still not observed or very weak, the user is to repeat the withdrawal step one more time before proceeding.

Once flow has been observed, the user is to secure device 1 to the patient using securement means 60. This is preferably around the ears or around the head and/or neck.

The foregoing disclosure of embodiments of the present invention has been presented for purposes of illustration and description. It is not intended to be exhaustive or to limit the invention to the precise forms disclosed. Many variations and modifications of the embodiments described herein will be obvious to one of ordinary skill in the art in light of the above disclosure. The scope of the invention is to be defined only by the claims, and by their equivalents.

What is claimed is:

1. A nasopharyngeal airway device comprising:
a flexible tubular body extending between a first distal end and a second proximal end, said first distal end configured for placement in a patient's pharynx via insertion through a nostril and said second proximal end configured to extend outside said patient's nose;
a first opening at the first distal end of said flexible tubular body, said first opening being offset from a longitudinal center of said flexible tubular body at said first distal end;
a second opening at the second proximal end of said flexible tubular body, said second opening being orthogonal to the longitudinal center of said flexible tubular body at said second proximal end.

2. The nasopharyngeal airway device of claim 1, wherein said flexible tubular body is formed with a shape-memory curvature between the first distal end and second proximal end, and the first opening at the first distal end of said flexible tubular body is offset toward an interior of said curvature.

3. The nasopharyngeal airway device of claim 1, wherein said flexible tubular body has an annular cross-section at the first distal end circumscribing a circular interior, and the first opening is an oblong having an area greater than the circular interior.

4. The nasopharyngeal airway device of claim 1, wherein said flexible tubular body has a rounded distal wall section adjoining the first opening.

5. The nasopharyngeal airway device of claim 1, wherein said flexible tubular body has an annular cross-section at the second proximal end circumscribing a circular interior, and the second opening is circular with an area greater than or equal to an area of the circular interior of the second proximal end.

6. The nasopharyngeal airway device of claim 1, wherein said flexible tubular body is stepped along its length between the first distal end and the second proximal end resulting in a distal segment having a smaller diameter than a proximal segment.

7. The nasopharyngeal airway device of claim 6, further comprising a plurality of radially-oriented openings along the distal segment of said flexible tubular body.

8. The nasopharyngeal airway device of claim 7, wherein said plurality of radially-oriented openings are spaced longitudinally from said first distal opening.

9. The nasopharyngeal airway device of claim 7, wherein said plurality of radially-oriented openings are spaced circumferentially about said first distal opening.

10. The nasopharyngeal airway device of claim 7, wherein said plurality of radially-oriented openings each have a diameter less than or equal to a diameter of the first circular cross-section of said tubular body.

11. The nasopharyngeal airway device of claim 5, wherein said second proximal end of the flexible tubular body is configured for attachment to an existing adjunct airway device.

12. The nasopharyngeal airway device of claim 6, further comprising an adjustable flange configured to slidably translate along said distal segment of said flexible tubular body.

13. The nasopharyngeal airway device of claim 12, wherein said adjustable flange includes means for controlling slidable friction along said proximal segment of said flexible tubular body.

14. The nasopharyngeal airway device of claim 12, wherein said adjustable flange includes at least one securement strap.

15. The nasopharyngeal airway device of claim 12, wherein said adjustable flange includes at least one depressed region configured to promote a sturdy grip.

16. The nasopharyngeal airway device of claim 12, wherein said distal segment of said flexible tubular body comprises at least one adjustment indicator configured to index a position of said adjustable flange.

17. The nasopharyngeal airway device of claim 12, wherein said adjustable flange further comprises protrusions at the interface with said tubular body.

18. The nasopharyngeal airway device of claim 12, wherein said adjustable flange further comprises at least one gripping extension that is laterally or axially oriented with respect to said tubular body.

19. The nasopharyngeal airway device of claim 12, wherein said adjustable flange further comprises a protrusion configured for fitting around part of a patient's nose.

20. The nasopharyngeal airway device of claim 6 further comprising at least one indicator along the first segment of said tubular body to index orientation of said first distal opening.

21. The nasopharyngeal airway device of claim 14, wherein said at least one securing strap is an elastic loop.

22. The nasopharyngeal airway device of claim 12, wherein said adjustable flange comprises at least one lateral extension.

23. The nasopharyngeal airway device of claim 1, further comprising a rounded distal wall section adjoining the first opening, said rounded distal wall section having a reduced wall thickness relative to adjoining wall sections to promote atraumatic insertion.

24. The nasopharyngeal airway device of claim 1, wherein said flexible tubular body is transparent or translucent.

25. The nasopharyngeal airway device of claim 24, wherein the proximal segment of said flexible tubular body is transparent or translucent.

26. The nasopharyngeal airway device of claim 1 further comprising an external flange extending radially from said flexible tubular body.

27. The nasopharyngeal airway device of claim 14, further comprising at least one notch in said adjustable flange configured to allow the adjustable flange to pinch said flexible tubular body when secured to a patient with said securement strap.
